(19) United States
(12) Patent Application Publication
(10) Pub. No.: US 2017/0035845 A1
(43) Pub. Date: Feb. 9, 2017

(54) Compositions and Methods for Inhibiting Pro-Inflammatory Cytokine Gene Expression

(71) Applicant: The Regents of the University of California, Oakland, CA (US)

(72) Inventors: Michael E. Selsted, Pasadena, CA (US); Prasad Tongaonkar, Arcadia, CA (US)

(21) Appl. No.: 15/230,166

(22) Filed: Aug. 5, 2016

Related U.S. Application Data
(60) Provisional application No. 62/202,403, filed on Aug. 7, 2015.

(51) Int. Cl. A61K 38/17

(52) U.S. Cl. CPC A61K 38/1729

(57) ABSTRACT

θ-defensins, non-human macrocyclic peptides, have been found to reduce expression of genes encoding for various pro-inflammatory peptides (such as cytokines and chemokines) in a highly selective manner. Methods for treating inflammatory conditions utilizing a θ-defensin and/or a θ-defensin analog, methods for modifying (e.g. down regulating) gene expression for pro-inflammatory peptides in a subject in need thereof using a θ-defensin and/or a θ-defensin analog, methods for selectively modifying expression of such genes and/or reducing pro-inflammatory peptides in a subject without inducing or worsening immunosuppression using a θ-defensin and/or a θ-defensin analog are discussed, as are compositions that include a θ-defensin and/or a θ-defensin analog in an amount and/or form suitable for use in such methods.

This application claims priority to U.S. Provisional Application No. 62/202,403 filed on Aug. 7, 2015. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

This invention was made with government support under Grant No. AI022931 awarded by the National Institute of Health. The government has certain rights in the invention.

### FIELD OF THE INVENTION

The field of the invention is methods and compositions effective in reducing the effect of pro-inflammatory cytokines, specifically inhibiting expression of cytokine genes.

### BACKGROUND

The background description includes information that may be useful in understanding the present invention. It is not an admission that any of the information provided herein is prior art or relevant to the presently claimed invention, or that any publication specifically or implicitly referenced is prior art.

Inflammation is a complex protective response to pathogens, tissue damage, and exposure to irritants, involving alteration in blood vessels, mobilization of immune cells, and release of a variety of chemical and peptide mediators. While inflammation serves to remove the initial cause of cell injury and eliminate necrotic cells from damaged tissue, the inflammatory response can itself be damaging. For example, chronic inflammation resulting from autoimmune disease can contribute to damage of the affected tissue. Similarly, inflammation resulting from acute processes, such as viral or bacterial infection, can result in tissue damage and septic shock. In addition the pain and swelling that accompany inflammation can be debilitating, particularly when it is the result of chronic conditions.

Unfortunately, current methods for treating inflammation suffer from a number of drawbacks. For example, traditional pharmaceutical approaches (e.g. treatment with steroids or non-steroidal inflammatory drugs) provide only short term relief, and often do so at the cost of significant side effects that limit the use of such drugs. More recently, “biologics” (for example, humanized monoclonal antibodies to proinflammatory cytokines) have been used to treat certain chronic conditions characterized by inflammation, however such approaches necessarily target only a single inflammation mechanism, and can result in immune suppression or even an immunocompromised state in a treated individual.

Mammalian defensins are cationic, tri-disulfide-containing peptides comprising three subfamilies denoted as α-, β-, and θ-defensins. Defensins contribute to host defense as antimicrobial agents (Ericksen et at, Antimicrob Agents Chemother 49:269-275 (2005)) and by regulating inflammatory (Khine et at, Blood 107:2936-2942 (2006)) and adaptive immune responses (Chertov et at, J Biol Chem 271:2935-2940 (1996)). All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. The α- and β-defensins have similar three dimensional topologies but differ in their disulfide linkages (Selsted and Ouellette, Nat Immunol 6:551-557 (2005)). θ-defensins are macrocyclic peptides that are expressed in Old World monkeys (e.g., macaques and baboons) and are the only known cyclic proteins in animals (Lehrer et at, J Biol Chem 287:27014-27019 (2012)). This basic θ-defensin backbone structure is produced by head-to-tail splicing of two nonapeptides that are excised from α-defensin-like precursors (Tang et al, Science 286:498-502 (1999)). In rhesus macaques, alternate binary splicing of nonapeptides encoded by three precursor genes provides six θ-defensin isoforms, rhesus theta-defensins (RTDs) 1-6 (Tang et at, Science 286:498-502 (1999); Leonova et at, J Leukoc Biol 70:461-464 (2001)). In baboons, alternate nonapeptide splicing produces ten θ-defensin isoforms (Garcia et at, Infect Immun 76:5883-5891 (2001)). θ-defensins are expressed at high levels in granules of neutrophils and in monocytes. These θ-defensins play a major role in the antimicrobial activities of rhesus neutrophil granule extracts. The RTD-1 isoform is the most abundant θ-defensin in macaques, constituting approximately 55% of the total θ-defensin content of rhesus neutrophils (Tongaonkar et al, J Leukoc Biol 89:283-290 (2011)).

Humans and other hominids lack θ-defensins due to the presence of a stop codon in the prepro-coding sequence of θ-defensin genes in these species (Nguyen et at, Peptides 24:1647-1654 (2003)). It has been suggested that the expression of θ-defensins in Old World monkeys is related to differences in immune and inflammatory responses of these nonhuman primates from those of humans (Leher et at, J Biol Chem 287:27014-27019 (2012)).

While α-, β-, and θ-defensins were initially identified on the basis of their broad spectrum antimicrobial properties, subsequent studies have disclosed immune regulatory roles for these peptides (Yang et al, Annu Rev Immunol 22:181-215 (2004)). For example, some α- and β-defensins are chemotactic for T cells, neutrophils, dendritic cells, and monocytes (Chertov et at, J Biol Chem 271:2935-2940 (1996); Yang et at, Science 286:525-528 (1999); Grigat et at, J Immunol 179:3958-3965 (2007); Soruri et at, Eur J Immunol 37:2474-2486 (2007)), and they induce secretion of proinflammatory cytokines from activated dendritic cells, peripheral blood mononuclear cells and epithelial cells (Khine et at, 107:2936-2942 (2006); Boniotto et at, Antimicrob Agents Chemother 50:1433-1441 (2006); Ito et at, Tohoku J Exp Med 227:39-48 (2012); Yin et al, Immunol 11:37. (2010); Niyonsaba et al, J Immunol 175:1776-1784 (2005); Li et at, Invest Ophthalmol Vis Sci 50:644-653 (2009); Syeda et al, J Cell Physiol 214:820-827 (2008)). In contrast to these pro-inflammatory activities, it has recently been reported that θ-defensins have anti-inflammatory properties both in vitro and in vivo. For example, RTD-1 was found to be a potent inhibitor of cytokine secretion by human peripheral blood leukocytes stimulated with diverse Toll-like receptor (TLR) agonists (Schaal et at, PLoS One 7, e51337 (2012)). Naturally occurring θ-defensin isoforms (RTDs 1-6) possess variable potency in reducing TNF in LPS- or E. coli-stimulated leukocytes. (Schaal et al, PLoS One 7, e51337 (2012)). RTD-1 has also been found to reduce inflammatory cytokines, including TNF-α, IL-1β and several chemokines in mouse models of SARS corona virus infection (Wohlford-Lenane et al, J Virol 83:11385-11390 (2009), in E. coli peritonitis, and in polymicrobial sepsis (Schaal et at, PLoS One 7, e51337 (2012)). Such responses, however, appear to be restricted to specific cytokines, and the mechanism of action is not clear.

Thus, there is still a need for methods and compositions that provide relief of inflammation through suppression of a range or plurality of pro-inflammatory moderators.

### SUMMARY OF THE INVENTION

The inventive subject matter provides apparatus, systems and methods in which a θ-defensin and/or a θ-defensin analog is provide in amounts and in a form that provides suppression of the expression of one or more genes encoding for a pro-inflammatory peptide(s), such as a cytokine and/or chemokine. Suitable θ-defensins include RTD-1 (SEQ ID NO. 1), RTD-2 (SEQ ID NO. 6), RTD-4 (SEQ ID NO. 7), RTD-5 (SEQ ID NO. 8), and RTD-6 (SEQ ID NO. 9). Embodiments of the inventive concept include methods for treating inflammatory conditions utilizing a θ-defensin and/or a θ-defensin analog, methods for modifying (e.g. down regulating) gene expression for pro-inflammatory peptides in a subject in need thereof using a θ-defensin and/or a θ-defensin analog, methods for selectively modifying expression of such genes and/or reducing pro-inflammatory peptides in a subject without inducing or worsening immunosuppression using a θ-defensin and/or a θ-defensin analog, and compositions that include a θ-defensin and/or a θ-defensin analog in an amount and/or form suitable for use in such methods.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 depicts typical data demonstrating that RTD-1 inhibits secretion of pro-inflammatory cytokines. TNF-α, IL-1β and IL-8 secreted in the medium were quantified by ELISA. Left panels. Medium from human monocytes stimulated for 4 hours with 20 ng/ml LPS or from unstimulated cells +/−RTD-1 (10 μg/ml). Results are from one of two similar, independent experiments. Right panels. Medium from differentiated THP-1 cells treated with LPS (100 ng/ml) for 2 hours or from untreated cells +/−RTD-1 (10 μg/ml). Results shown are mean+/−SD from 3 independent experiments. # indicates cytokine was below detection limit.

FIG. 2 depicts typical data demonstrating that RTD-1 down regulates mRNA of pro-inflammatory cytokines. Real time PCR analysis of cDNA from differentiated THP-1 cells treated with LPS (100 ng/ml)+/−RTD-1 (10 μg/ml) for 2 hours or from control untreated cells. Results shown are mean+/−SD from 3 independent experiments.

FIGS. 3A to 3E depict typical data demonstrating that RTD-1 inhibits NF-κB activation in cells stimulated with TLR agonists. SEAP activity in the medium of cells stimulated with TLR agonists in presence or absence of RTD-1 was assayed using Quantiblue. SEAP activity is expressed as fold induction compared with untreated control cells. In FIG. 3A differentiated THP-1 Dual cells were treated with or without LPS (100 ng/ml) in the presence or absence of 10 μg/ml RTD-1. In FIG. 3B cells were treated with or without LPS (100 ng/ml) and in the presence or absence of either RTD-1 or HNP-1 (numbers indicate concentrations of RTD-1 and HNP-1 in μg/ml; 2.4 and 4.8 μM correspond to 5 and 10 μg/ml RTD-1 and 8.25 and 16.5 μg/ml HNP-1 respectively). In FIG. 3C the upper panel shows the structures of RTD-1 and S7 peptide where the dotted lines indicate disulfide linkage, whereas the lower panel shows data from THP-1 Dual cells stimulated with 100 ng/ml LPS in the presence or absence of 10 μg/ml RTD-1 or the acyclic S7 peptide. FIG. 3D shows results from differentiated THP-1 Dual cells treated with Pam3CSK4 (25 ng/ml) in the presence or absence of 10 μg/ml RTD-1. FIG. 3E shows results from HEK-Blue hTLR9 cells treated with or without ODN2006 (ODN) and +/−10 μg/ml RTD-1. The numbers indicate the concentration of ODN2006 in μg/ml. Results are shown as means+/−SD from 3 independent experiments. * P<0.05 when compared to treatment with agonist alone.

FIGS. 4A and 4B depict typical data demonstrating that Inhibition of NF-κB DNA-binding activity by RTD-1. Nuclear extracts containing 2.8 μg protein from control differentiated THP-1 cells and stimulated with (in FIG. 4A) LPS (100 ng/ml), or (in FIG. 4B) TNF-α (2 ng/ml) and with or without 10 μg/ml RTD-1 were assayed for DNA-binding. Results are means+/−SD from 3 independent experiments. * P<0.05; compared to LPS or TNF-α treated samples. Lower panels of FIG. 4A and FIG. 4B show Western blots of respective THP-1 nuclear extracts containing 8.1 μg protein used for DNA-binding assay probed with anti-NF-κB p65 antibodies.

FIGS. 5A and 5B depict typical data demonstrating differential effects of RTD-1 and the TACE inhibitor marimastat (MM). FIG. 5A shows results from studies where THP-1 macrophages were treated with or without LPS (100 ng/ml) in the presence or absence of MM and RTD-1. TNF-α (black bars) and IL-1β (open bars) secreted in the medium were then measured by ELISA. Results are means+/−SD from 3 independent experiments. FIG. 5B shows results from studies where differentiated THP-1 Dual cells were stimulated with 100 ng/ml LPS and co-incubated with the indicated reagents and NF-κB activity was measured relative to control using the Quantiblue assay. Results shown are means+/−SD from 3 independent experiments. * P<0.05 when compared to MM+LPS treatment.

FIG. 6 depicts typical data demonstrating that RTD-1 stabilizes IκBα and blocks phosphorylation of p38 MAP and JNK kinases. Cell extracts (15 μg protein) were obtained from THP-1 cells stimulated with diluent, 100 ng/ml LPS, LPS+10 μg/ml RTD-1, or RTD-1 alone. Extracts were resolved on SDS-tricine gels and western blots were probed with anti-IκBα, anti-Phospho p38 MAP kinase and anti-Phospho SAP/JNK kinase antibodies.

FIG. 7 depicts typical data demonstrating that RTD-1 modulates phosphorylation of multiple inflammatory signaling proteins. Extracts from control, 100 ng/ml LPS treated, 100 ng/ml LPS+10 μg/ml RTD-1 treated, or 10 μg/ml RTD-1 treated THP-1 cells were used to probe a Phospho-MAPK array. Positive controls were spotted at (A1, A2), (A21, A22) and (F1, F2) and negative control at (E19, E20). Dot intensities from two independent experiments were quantified with ImageJ software, normalized and the mean values were plotted (lower panel). RTD-1 suppressed LPS induced phosphorylation of 7 proteins implicated in LPS-induced inflammatory signaling. Treatment of THP-1 macrophages with peptide alone increased Akt phosphorylation.

FIGS. 8A to 8C depict typical data demonstrating that RTD-1 stimulation of Akt phosphorylation. In FIG. 8A THP-1 cells were treated with diluent or 10 μg/ml RTD-1 and extracts were prepared from cells harvested at the indicated times. Extracts were resolved on SDS-tricine gels and probed using anti-Phospho-Akt (P-Akt) and anti-Akt antibodies (upper panel). Band intensities were quantified with ImageJ software, normalized relative to total Akt, and the average fold-increase of P-Akt from two independent experiments was plotted as function of incubation time (lower panel). In FIG. 8B THP-1 macrophages were incubated with 10 ug/ml of RTD-1 or diluent+/−10 μM Ly294002. Western blots were performed using Akt or P-Akt antibodies. In FIG. 8C THP-1 Dual cells were pretreated with Ly294002 for 60 minutes and then stimulated overnight with 100 ng/ml LPS in the presence of 10 μg/ml RTD-1. Medium was analyzed for SEAP activity and fold stimulation was calculated for each Ly294002 concentration relative to cells treated with no Ly294002. Results are mean+/−SEM from four independent experiments and the effect of Ly294002 treatment on SEAP expression was significant (P<0.05) at all concentrations tested.

FIG. 9 depicts typical data demonstrating the effect of RTD-1 on signaling pathways in human monocytes. Extracts (˜7 μg protein) from unstimulated monocytes or monocytes stimulated with 100 ng/ml LPS+/−RTD-1 (10 μg/ml) for 30 minutes were resolved on SDS-Tricine gels and the immunoblots were probed with antibodies against IκBα, Phospho-p38 MAP kinase and Phospho-Akt.

FIG. 10 schematically depicts immunoregulatory effects of RTD-1. The schematic shows the suppression of NF-κB activation stimulated by extracellular (TLR 1/2 & 4) and intracellular (TLR9) receptors, inhibitory and stimulatory effects of RTD-1 on LPS-stimulated pathways, and negative regulatory pathways mediated by P-Akt.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

The inventive subject matter provides compositions and methods in which a θ-defensin (for example RTD-1 (SEQ ID NO. 1) and/or an RTD-1 analog, derivative, or mutant) is used to suppress expression of genes encoding two or more pro-inflammatory moderators. Such pro-inflammatory moderators can include cytokines and/or chemokines. This gene suppression results in a reduction or elimination of inflammatory processes across multiple inflammatory pathways.

Various objects, features, aspects and advantages of the inventive subject matter will become more apparent from the following detailed description of preferred embodiments, along with the accompanying figures.

One should appreciate that the disclosed techniques provide many advantageous technical effects including suppression of multiple pro-inflammatory mediators with a single compound. In addition, specific suppression of such pro-inflammatory mediators at the gene expression level is highly selective compared to current treatment modalities and can result in reduced side effects. Such an approach can also provide an extended duration of the effect of a single dose relative to prior art approaches,

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Abbreviations and acronyms used throughout this application include:
BCA Bicinchoninic Acid
CCL Chemokine (C-C motif) ligand
CLP Cecal ligation and puncture
CREB cAMP response Element Binding protein
CT Cycle Threshold
DMSO Dimethyl Sulfoxide
ERK Extracellular signal Regulated Kinase
FBS Fetal Bovine Serum
gDNA Genomic DNA
HI Heat Inactivated
HOAc Acetic acid
HNP Human neutrophil peptide
HSP Heat shock protein
IL Interleukin
IKK IκB kinase
LPS Lipopolysaccharide
MAP kinase Mitogen activated protein kinase
MM Marimastat
NF-κB Nuclear Factor kappa
ODN Oligo deoxynucleotide
PBS Phosphate buffered saline
PCR Polymerase Chain Reaction
PI3K Phosphatidyl inositol 3 phosphate kinase
PMA Phorbol 12-myristate 13-acetate
PMSF Phenyl methyl sulfonyl fluoride
PPC Positive PCR control
RTC Reverse transcription control
RTD Rhesus theta defensin
SAPK/JNK Stress activated protein kinase/Jun N-terminal kinase
SEAP Secreted embryonic alkaline phosphatase
TACE/ADAM17 Tumor necrosis factor alpha converting enzyme/A disintegrin and metalloproteinase domain 17
TAK1 TGF β activated kinase 1
TLR Toll like receptor
TNF Tumor necrosis factor

Studies on θ-defensins have revealed the pleiotropic properties of these macrocyclic host defense molecules. Initially isolated based on their antibacterial and antifungal properties in vitro, subsequent studies have demonstrated broader host defense properties mediated by their antiviral properties against Herpes Simplex virus, HIV and influenza, and arming of phagocytes for enhanced killing of Bacillus anthracis. Protective effects of systemically administered RTD-1 are observed in mouse models of SARS coronavirus infection and sepsis. To gain insights into the immunomodulatory mechanisms mediated by θ-defensins, inventor(s) analyzed the effects of RTD-1 on inflammatory pathways in THP-1 macrophages and have, surprisingly, identified a previously unknown mode of action.

The inventor contemplates that properties identified as being associated with RTD-1 and/or other θ-defensins are to be found in analogs of these cyclic peptides. An analog of a θ-defensin can have greater than 70%, 75%, 80%, 85%, 90%, 95%, or more amino acid sequence identity to a corresponding parent θ-defensin. Suitable analogs can be based on RTD-1 (SEQ ID NO. 1), RTD-2 (SEQ ID NO. 5), RTD-3 (SEQ ID NO. 6), RTD-4 (SEQ ID NO. 7), RTD-5 (SEQ ID NO. 8), and/or RTD-6 (SEQ ID NO. 9) as parent θ-defensins. Differences in amino acid sequence can be produced by deletion, addition, and/or substitution of one or more amino acids. Such substitutions can be conservative (i.e. replacement with an amino acid having similar charge, size, reactivity, and/or hydrophobicity) or non-conservative (i.e. replacement with an amino acid having a different charge, size, reactivity, and/or hydrophobicity). Such analogs can incorporate non-naturally occurring amino acids, amino acid analogs, and/or amino acid modifications via conjugation (such as PEGylation). Such analogs can be truncated versions of a θ-defensin in which one or more amino acids of the parent θ-defensin have been deleted, but that retain essential conformation and structure. For example, the synthetic cyclic peptides designated RTD 1-27 (SEQ ID NO. 2), RTD 1-28 (SEQ ID NO. 3), and/or RTD 1-29 (SEQ ID NO. 4) can be considered analogs of RTD-1 (SEQ ID NO. 1). Similarly, analogs of RTD-2 (SEQ ID NO. 5), RTD-3 (SEQ ID NO. 6), RTD-4 (SEQ ID NO. 7), RTD-5 (SEQ ID NO. 8), and RTD-6 (SEQ ID NO. 9) can be used in embodiments of the inventive concept. In some embodiments such a θ-defensin analog can have improved pharmacological properties (e.g. solubility, serum half-life, bioavailability, oral or mucosal absorption, etc.) relative to the related parent θ-defensin.

One embodiment of the inventive concept is a method for treating an acute and/or chronic inflammatory condition by inhibition of the gene expression of one, two, or more than two pro-inflammatory peptides (e.g. cytokines), where such inhibition is provided by application of a θ-defensin and/or θ-defensin analog to a subject in need of anti-inflammatory treatment. Dosage and dosing frequency of the θ-defensin and/or θ-defensin analog can be selected to provide relief from inflammation and/or measurable reduction in pro-inflammatory peptide expression in less than 10 minutes, less than 15 minutes, less than 20 minutes, less than 30 minutes, less than 1 hour, less than 2 hours, less than 3 hours, less than 4 hours, less than 6 hours, less than 8 hours, less than 12 hours, less than 16 hours, and/or less than 24 hours from administration of an initial dose. Relief from inflammation and/or reduction in pro-inflammatory peptide expression can persist for at least 1 hour, at least 2 hours, at least 4 hours, at least 6 hours, at least 8 hours, at least 12 hours, at least 16 hours, at least 24 hours, at least 48 hours, at least 72 hours, and/or more than 72 hours from establishment of the anti-inflammatory or reduction of pro-inflammatory peptide expression effect. Dosing schedules can be selected for dosing intervals ranging from three times a day to once a week. In some embodiments the θ-defensin and/or θ-defensin analog can be provided as a constant infusion. Acute inflammatory conditions that can be treated in this manner are characterized by sudden and severe onset, and include sepsis, traumatic injury, thermal burns, chemical burns, electrical burns, radiation burns, acute allergic reactions, and inhalation injuries. Chronic inflammatory conditions that can be treated in this manner are characterized by gradual onset and persistence over time, and include rheumatoid arthritis, ankylosing spondylitis, inflammatory bowel disease, ulcerative colitis, autistic enterocolitis, psoriasis, psoriatic arthritis, Crohn's disease, Behcet's disease, lupus, hidradenitis suppurativa, refractory asthma, colitis, dermatitis, diverticulitis, hepatitis, nephritis, Parkinson's disease, Alzheimer's disease, Huntington's disease, congestive heart disease, atherosclerosis, and uveitis.

Another embodiment of the inventive concept is a method of inhibiting gene expression of one, two, or more than two pro-inflammatory peptides (e.g. cytokines), where such inhibition is provided by application of a θ-defensin and/or θ-defensin analog to a subject in need of such a reduction in gene expression. Such a subject can, for example, be in need of anti-inflammatory treatment. Dosage and dosing frequency of the θ-defensin and/or θ-defensin analog can be selected to provide a measurable reduction in pro-inflammatory peptide expression in less than 10 minutes, less than 15 minutes, less than 20 minutes, less than 30 minutes, less than 1 hour, less than 2 hours, less than 3 hours, less than 4 hours, less than 6 hours, less than 8 hours, less than 12 hours, less than 16 hours, and/or less than 24 hours from administration of an initial dose. The reduction in pro-inflammatory peptide expression can persist for at least 1 hour, at least 2 hours, at least 4 hours, at least 6 hours, at least 8 hours, at least 12 hours, at least 16 hours, at least 24 hours, at least 48 hours, at least 72 hours, and/or more than 72 hours from establishment of the reduction of pro-inflammatory peptide expression effect. Dosing schedules can be selected for dosing intervals ranging from three times a day to once a week. Such a reduction in gene expression can result in gene expression levels that are similar or identical to expression levels of pro-inflammatory peptides (e.g. cytokines) in the absence of an inflammatory stimulus. In some embodiments the θ-defensin and/or θ-defensin analog can be provided as a constant infusion. Such a reduction in gene expression can result in gene expression levels that are similar or identical to expression levels of pro-inflammatory peptides (e.g. cytokines) in the absence of an inflammatory stimulus. An inflammatory stimulus can be an acute inflammatory condition characterized by sudden and severe onset, and include sepsis, traumatic injury, thermal burns, chemical burns, electrical burns, radiation burns, acute allergic reactions, and inhalation injuries. Similarly, an inflammatory stimulus can be a chronic inflammatory condition characterized by gradual onset and persistence over time, and include rheumatoid arthritis, ankylosing spondylitis, inflammatory bowel disease, ulcerative colitis, autistic enterocolitis, psoriasis, psoriatic arthritis, Crohn's disease, Behcet's disease, lupus, hidradenitis suppurativa, refractory asthma, colitis, dermatitis, diverticulitis, hepatitis, nephritis, Parkinson's disease, Alzheimer's disease, Huntington's disease, congestive heart disease, atherosclerosis, and uveitis.

Another embodiment of the inventive concept is a composition that includes a θ-defensin and/or θ-defensin analog in an amount and form suitable for reducing expression of one, two, or more than two pro-inflammatory peptide(s) (e.g. cytokines). Such a composition can be in the form of a solid, powder, liquid, gel, and/or suspension. Such a composition can be formulated to be administered by injection, infusion, orally, rectally, vaginally, as an inhalant (e.g. a mist or powder), and/or topically. The θ-defensin and/or θ-defensin analog can be provided to provide a dose ranging from 1 μg to 10 mg per kg of body weight of an individual to be treated. Such compositions can also include additional components that do not have direct activity in reducing inflammation and/or expression of a gene encoding a pro-inflammatory peptide, such as excipients, flavorants, stabilizers, and/or bulking agents. In some embodiments the composition can include pharmaceutically active compounds that are useful in co-therapy, for example an antibiotic, antihistamine, bronchodilator, steroid, and/or non-steroidal anti-inflammatory compound.

Another embodiment of the inventive concept is the use of a θ-defensin and/or θ-defensin analog in the formulation of a medicament useful for treating an acute and/or chronic inflammatory condition by inhibition of the gene expression of one, two, or more than two pro-inflammatory peptides (e.g. cytokines), where such inhibition is provided by application of a θ-defensin and/or θ-defensin analog to a subject in need of anti-inflammatory treatment. Dosage and dosing frequency of the θ-defensin and/or θ-defensin analog can be selected to provide relief from inflammation and/or measurable reduction in pro-inflammatory peptide expression in less than 10 minutes, less than 15 minutes, less than 20 minutes, less than 30 minutes, less than 1 hour, less than 2 hours, less than 3 hours, less than 4 hours, less than 6 hours, less than 8 hours, less than 12 hours, less than 16 hours, and/or less than 24 hours from administration of an initial dose. Relief from inflammation and/or reduction in pro-inflammatory peptide expression can persist for at least 1 hour, at least 2 hours, at least 4 hours, at least 6 hours, at least 8 hours, at least 12 hours, at least 16 hours, at least 24 hours, at least 48 hours, at least 72 hours, and/or more than 72 hours from establishment of the anti-inflammatory or reduction of pro-inflammatory peptide expression effect. Dosing schedules can be selected for dosing intervals ranging from three times a day to once a week. In some embodiments the θ-defensin and/or θ-defensin analog can be provided as a constant infusion. Acute inflammatory conditions that can be treated in this manner are characterized by sudden and severe onset, and include sepsis, traumatic injury, thermal burns, chemical burns, electrical burns, radiation burns, acute allergic reactions, and inhalation injuries. Chronic inflammatory conditions that can be treated in this manner are characterized by gradual onset and persistence over time, and include rheumatoid arthritis, ankylosing spondylitis, inflammatory bowel disease, ulcerative colitis, autistic enterocolitis, psoriasis, psoriatic arthritis, Crohn's disease, Behcet's disease, lupus, hidradenitis suppurativa, refractory asthma, colitis, dermatitis, diverticulitis, hepatitis, nephritis, Parkinson's disease, Alzheimer's disease, Huntington's disease, congestive heart disease, atherosclerosis, and uveitis.

It should be appreciated that θ-defensins are only found in non-human primates. As such, presence of a θ-defensin or θ-defensin analog in a human being cannot be considered a naturally-occurring phenomena.

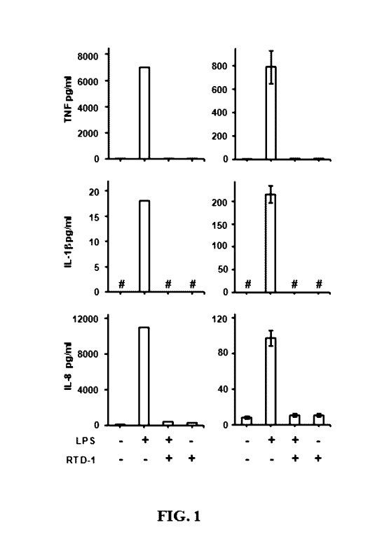

RTD-1 inhibits secretion of several proinflammatory cytokines by human buffy coat cells stimulated with E. coli cells as well as with agonists for TLR2, 4, 5, and 8 with particularly marked suppression of TNF-α, IL-1α/β, IL-6, IL-8, and CCL3, and CCL4. Consistent with these findings, 10 μg/ml of RTD-1, a concentration that the inventors have found is effective in blocking TNF secretion from stimulated blood cells, was also found to be highly effective in blocking secretion of TNF-α, IL-1β, and IL-8 from LPS stimulated THP-1 cells and human monocytes (see FIG. 1). RTD-1 (SEQ ID NO. 1) binds inefficiently to LPS; as a result its immune regulatory effects are not mediated by neutralization of LPS. Surprisingly, the inventors have found that RTD-1 suppression of these cytokines, and of CCL3 and CCL4 is due to down regulation of the corresponding mRNA levels. This indicates that at least one anti-inflammatory mechanism of θ-defensins is through regulation of inflammatory gene expression. It should be appreciated that this effect is selective, as RTD-1 (SEQ ID NO. 1) alone does not affect cytokine mRNA or protein levels in unstimulated cells (see FIGS. 1 and 2).

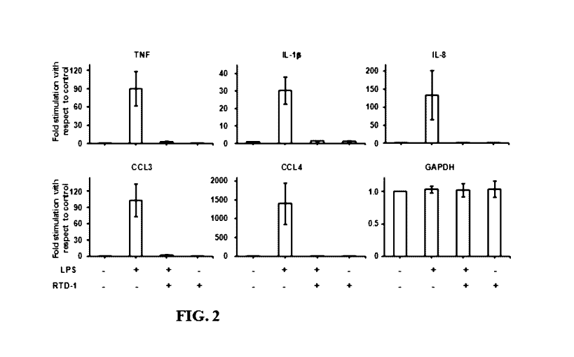

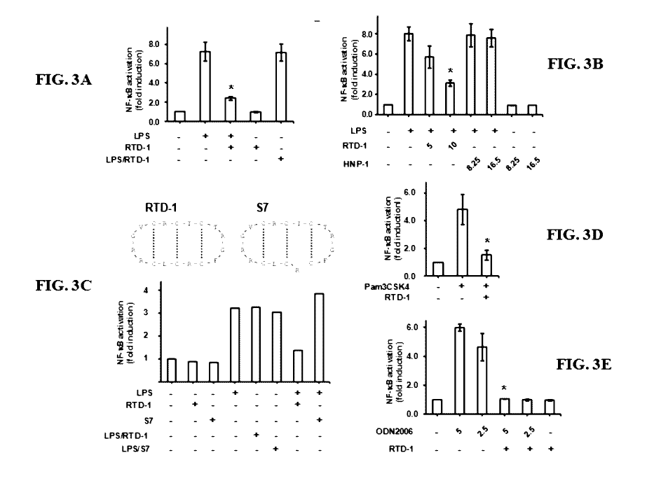

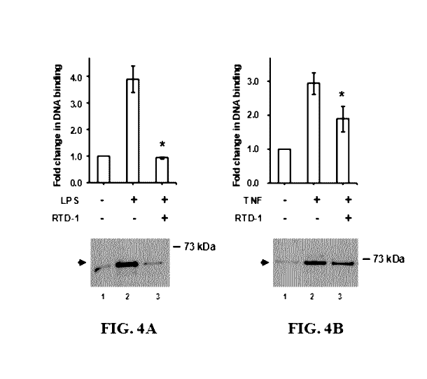

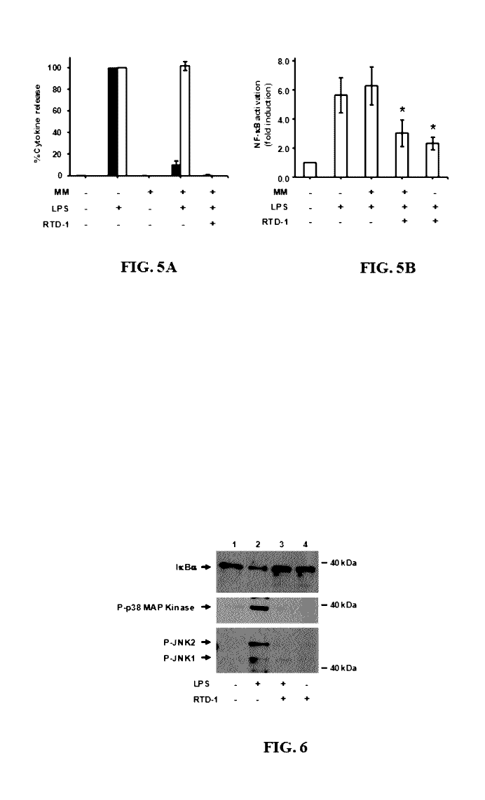

The inventors also analyzed the effect of θ-defensin peptide treatment on NF-κB activation in THP-1 cells treated with LPS and Pam3CSK4, agonists for TLRs 4, 1/2 (cell surface) respectively, and in HEK Blue hTLR9 cells treated with ODN2006, agonist for TLR9 (intracellular). Surprisingly, RTD-1 treatment markedly inhibited NF-κB activation by each agonist (see FIGS. 3A, 3B, 3C, 3D, and 3E). This inhibitory effect is dose-dependent. RTD-1 was found to inhibit translocation of NF-κB p65 to the nucleus in LPS stimulated macrophages (see FIGS. 4A and 4B) and stabilized IκBα (see FIG. 6). Notably, the human α-defensin HNP-1 had no inhibitory activity (see FIG. 3B). It should be appreciated that an acyclic form of RTD-1 (S7; see FIG. 3C) did not inhibit NF-κB activation, demonstrating that the cyclic structure of this macrocyclic peptide is essential for this activity.

Previously, inventor(s) showed that the RTD-1-mediated blockade of TNF-α secretion by E. coli stimulated whole blood was extremely rapid. Inventors found that RTD-1 (SEQ ID NO. 1) reduced TNF-α-mediated NF-κB activation in THP-1 cells, but to a lesser extent than the peptide's effect on LPS stimulation (see FIGS. 4A and 4B). Similarly, RTD-1 suppressed both TNF-α and IL-1β secretion by LPS-treated THP-1 cells. This is in contrast to the effects of marimastat (see FIG. 5), a potent inhibitor of the TACE/ADAM17 sheddase that generates soluble TNF-α and which did not inhibit IL-1β release. Thus blockade of TNF-α release, and subsequent suppression of autocrine signaling, cannot alone account for the immunomodulatory effects of RTD-1 (SEQ ID NO. 1).

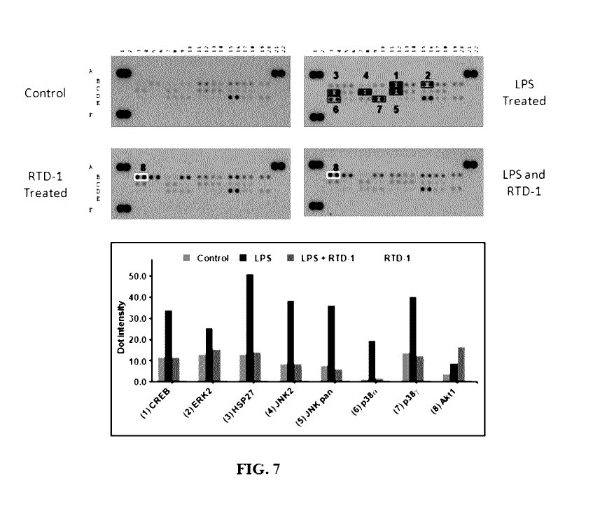

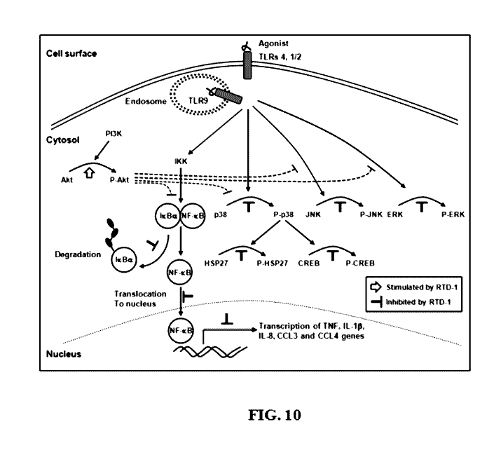

Inventors also analyzed the effect of RTD-1 (SEQ ID NO. 1) on signaling kinases implicated in LPS-induced inflammatory responses in macrophages. RTD-1 (SEQ ID NO. 1) inhibited LPS-induced phosphorylation of several inflammatory signaling proteins including p38, JNK, ERK2, CREB and H5P27 (see FIGS. 6 and 7). Inhibition of CREB and H5P27 phosphorylation is consistent with the suppression of p38 MAP kinase which phosphorylates these proteins (see FIG. 10). The p38 MAP kinase is implicated in stabilization of mRNAs containing AU-rich elements of immune response genes such as TNF-α, IL-6, CCL3, IL-8, and other immune response genes. The MEK-ERK pathway is involved in nucleocytoplasmic transport of TNF-α mRNA in mouse macrophage cells. Stimulation of JNK MAP kinase leads to activation of the AP-1 class of transcription factors which are involved in activation of immune response genes. The inventors have found that, surprisingly, RTD-1 (SEQ ID NO. 1) regulates inflammatory signaling by inhibiting the phosphorylation of several inflammatory signaling proteins, for example signaling proteins involved in these pathways.

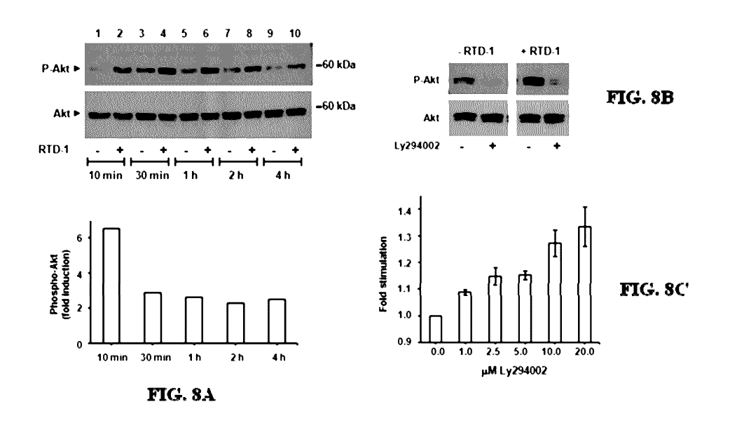

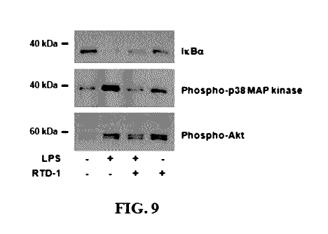

THP-1 cell exposure to RTD-1 alone was found to result in phosphorylation of Akt (see FIG. 7), which occurs very rapidly after RTD-1 treatment and produces a sustained effect (see FIGS. 8A, 8B, and 8C). Surprisingly, the inventors found that this effect is highly selective as RTD-1 did not increase baseline phosphorylation of other kinase targets evaluated in otherwise untreated cells (see FIGS. 7, 8A, 8B, and 8C). This effect is also found in other cell types, as RTD-1 (SEQ ID NO. 1) inhibited the degradation of IκBα, phosphorylation of p38 MAP kinase in LPS-stimulated human monocytes and also stimulated Akt phosphorylation in these cells (see FIG. 9).

This RTD-1 (SEQ ID NO. 1)-induced phosphorylation of Akt was blocked by a specific PI3K inhibitor, indicating that the PI3K-Akt pathway can be involved in mediating the anti-inflammatory effects of RTD-1 (SEQ ID NO. 1) (see FIGS. 8A, 8B, and 8C). PI3K-Akt is a negative regulator of NF-κB and MAP kinase pathways and inhibits downstream TNF-α gene expression in LPS stimulated cells. Inhibition of the PI3K-Akt pathway increases the mortality and circulating pro-inflammatory cytokine levels in endotoxemic mice as well as in mice with polymicrobial sepsis induced by cecal ligation and puncture (CLP). This suggests that a θ-defensin and/or θ-defensin analog can act through the PI3K-Akt pathway to reduce or eliminate release of pro-inflammatory peptides (such as pro-inflammatory cytokines), thereby reducing or eliminating an undesirable inflammatory response in a subject treated with such a θ-defensin and/or θ-defensin analog. Interestingly both human α-defensin (e.g. HNP1) and human β-defensin (e.g. HBD2 and HBD3) can stimulate expression and secretion of pro-inflammatory cytokines from human conjunctival epithelial cells, which is accompanied by phosphorylation of Akt. Both α- and β-defensins are known to regulate cellular responses through receptor mediated pathways. The human β-defensins, HBD2 and HBD3, induce chemotaxis in monocytes through the CCR2 receptor and HBD2 induces chemotaxis in dendritic and T cells through the CCR6 receptors. To date there have been no reports of a receptor for θ-defensins, suggesting that θ-defensin and/or θ-defensin analogs can utilize either heretofore undiscovered receptors for these non-human peptides or act in a receptor-independent fashion. Studies are ongoing to identify RTD-1 (SEQ ID NO. 1) targets upstream of the PI3K-Akt pathway and the relationship of RTD-1-induced Akt phosphorylation to downstream signaling pathways. It should be noted that the suppression of TNF-α secretion by RTD-1 (SEQ ID NO. 1) is extremely rapid suggesting that RTD-1 (SEQ ID NO. 1) can also regulate pathways distinct from those currently known to be involved in signaling and expression of pro-inflammatory genes.

Human β-defensin (hBD-3) inhibits LPS-induced gene transcription and pro-inflammatory cytokine secretion in mouse RAW264.7 macrophages via inhibition of the NF-κB pathway involving signaling through MyD88 and TRIF. It has been theorized that the contrasting pro- and anti-inflammatory properties reported for β-defensins may reflect differences in structural features and/or purity of different peptide preparations. Another possibility is that the proinflammatory versus anti-inflammatory properties of a single peptide can be a function of local concentration of the peptide. Without wishing to be bound by theory, the inventors theorize that θ-defensins, which are expressed at very high levels in granules of circulating neutrophils and monocytes, provide a physiologic dampening mechanism for inflammatory processes following their release and/or secretion, systemically and/or locally. As such θ-defensins and θ-defensin analogs can act as a safe and effective anti-inflammatory compound that can be applied to a wide variety of inflammatory conditions, both chronic and acute, and both systemic and localized.

In summary, RTD-1 (SEQ ID NO. 1) suppresses NF-κB activation and hence pro-inflammatory peptide (e.g. pro-inflammatory cytokine) expression and release. Such immunomodulation occurs at both the TLR and TNF pathways. Evidence for a role for induction of Akt phosphorylation is suggested by RTD-1 (SEQ ID NO. 1) induced P-Akt in both naïve and LPS-stimulated cells. The immunomodulatory properties of the macrocyclic peptide are distinct from those of human α-defensins, and RTD-1 (SEQ ID NO. 1) suppresses inflammation by mechanisms that are distinct from those of hBD-3. Further, since RTD-1, related θ-defensins, and θ-defensin differentially inhibit TNF secretion from stimulated blood cells, θ-defensin isoforms and θ-defensin analogs can discriminate between regulatory pathways that determine whether the subject response to inflammatory stimuli results in health or disease, and therefore can selectively provide anti-inflammatory effects to diseased and/or damaged tissue.

### Examples

### Reagents

Phorbol 12-myristate 13-acetate (PMA), marimastat, and phenyl methyl sulfonyl fluoride (PMSF) were from Sigma Chemical Co. (St. Louis, Mo.), E. coli K12 lipopolysaccharide (LPS), Pam3CSK4, ODN2006 and Quantiblue were from Invivogen (San Diego, Calif.). Anti-phospho-p38 MAP kinase, anti-IκBα, anti-phospho-SAPK/JNK, anti-NF-κB (p65), anti-phospho-Akt (Ser473) and anti-Akt pan antibodies were from Cell Signaling Technology (Danvers, Mass.). Marimastat and Ly294002 (Cell Signaling Technology) solutions were prepared at 1000× in DMSO. Carrier free TNF-α (Cell Signaling Technology) was suspended at 2 μg/ml in phosphate buffered saline (PBS) containing 5% heat inactivated (HI) fetal bovine serum (FBS). Synthetic RTD-1 (SEQ ID NO. 1) hydrochloride (>99%) was prepared as described in Tang et al, Science 286:498-502 (1999). Human α-defensin HNP-1 was purified from neutrophils. Stock solutions of RTD-1 (SEQ ID NO. 1), S7 peptide and HNP-1 were prepared in sterile 0.01% acetic acid. It should be appreciated that the S7 peptide is a linearized form of RTD-1 (SEQ ID NO. 1), and thus shares the same amino acid sequence while having an acyclic structure.

Cell Culture:

THP-1 monocytes (ATCC, Manassas, Va.) were cultured in RPMI 1640+10% FBS and penicillin/streptomycin (P/S). THP-1 Dual cells (Invivogen, San Diego, Calif.) which express the NF-κB reporter, secreted embryonic alkaline phosphatase (SEAP), were grown in RPMI 1640+10% HI-FBS and antibiotics. THP-1 cell differentiation was induced by treatment of cells (˜3.3×10⁵ cells/ml) with 100 nM PMA and cultured for 2 days. HEK-Blue™ hTLR9 cells (Invivogen) were grown in DMEM medium containing 10% HI-FBS, P/S and normocin. Cells were cultured at 37° C. in 5% CO₂. Cytokine assays. PMA-differentiated THP-1 cells (˜8×10⁵ cells) cultured in 6-well plates were washed twice with complete medium and grown in fresh medium containing 1% FBS for 24 hours. Cells were washed twice with fresh medium and incubated for 2 hours prior to further manipulation. Cells were stimulated with 100 ng/ml LPS in the presence or absence of 10 μg/ml RTD-1 or vehicle (0.01% acetic acid) for 2 hours. In some experiments, cells were first incubated for 1 hour with 10 μg/ml of marimastat or vehicle (DMSO), after which LPS and/or RTD-1 were added and incubated for 2 hours as above. Peripheral blood CD14+ monocytes (Lonza, Walkersville, Md.) were thawed, rinsed with RPMI 1640 containing 10% HI FBS, P/S and normocin and then resuspended in RPMI 1640 containing 1% HI FBS plus antibiotics as above (10⁶ cells/nil) for 2 hours. The cells were then stimulated for 4 hours as described in the legend. Medium was collected, clarified by centrifugation first at 250×g for 8 minutes and then at 5000×g for 5 minutes. TNF-α, IL-1β and IL-8 were quantified by ELISA (Life Technologies, Carlsbad, Calif.).

Real Time Quantitative PCR.

Differentiated THP-1 cells were treated with LPS and RTD-1 for 2 hours as above, washed with PBS and RNA was isolated using an RNA mini kit (Zymo Research, Irvine, Calif.) or with RNeasy mini kit (Qiagen, Valencia, Calif.). RNA integrity was confirmed on agarose gels and RNA samples with A260/280 and A260/230 ratios ≧1.7 were used to generate cDNA. Briefly, 400 ng RNA was incubated with genomic DNA (gDNA) elimination (GE) buffer for 5 minutes at 4° C. and reverse transcribed using the RT² first strand synthesis kit (Qiagen). Custom PCR arrays (SA Biosciences, Frederick, Md.) containing human genes of interest and controls (for reverse transcriptase and PCR efficiency, RTC and PPC respectively) were used for simultaneous real time PCR analysis. Master mix containing cDNA and RT² SYBR Green™ was added to array wells. PCR cycling parameters were: 1 cycle 10 minutes at 95° C., 40 cycles of 15 seconds at 95° C., 1 minute at 60° C. on a BioRad C1000 Thermal Cycler equipped with a CFX96 real time system. Melting curve analysis confirmed a single amplicon with CT PPC=20±2 and ACT (RTC-PPC)<5 for all samples. The ACTB gene was used for normalization of gene expression and fold stimulation (compared to untreated cells) was calculated using the ΔΔCT method. Absence of gDNA in cDNA samples (CT>35) was confirmed using a human gDNA primer (SA Biosciences).

NF-κB Activity Assay.

One ml aliquots of PMA-differentiated THP-1 Dual cells (3.3×10⁵ cells/nil) in 24 well tissue culture plates were starved for 1 day in complete medium with 1% HI FBS as described above. For experiments with S7 peptide, 0.5 ml cultures in 48 well plates were used. Fresh medium containing 1% HI FBS was added to the cells and after 2 hours cells were stimulated with 100 ng/ml LPS or 25 ng/ml Pam3CSK4 in the presence of RTD-1 or 0.01% acetic acid (HOAc, vehicle). In some experiments cells were pre-incubated with marimastat, Ly294002 or vehicle for 1 hour prior to addition of LPS and/or RTD-1 (SEQ ID NO. 1). Following overnight incubation, medium was clarified by centrifugation and SEAP activity was determined using Quantiblue reagent at 37° C. and measurement of absorbance at 625 nm. To test the effect of RTD-1 on TLR9-dependent NF-κB activation, 2×10⁵ HEK-Blue™ hTLR9 cells/ml were cultured for 1 day in 24 well plates and stimulated overnight with ODN2006 with or without RTD-1. Medium was harvested and SEAP activity was assayed using Quantiblue™ as above. NF-κB activation was defined as fold stimulation of SEAP activity with respect to control cells.

NF-κB DNA-Binding Activity.

Nuclear extracts were prepared (nuclear extraction kit; Cayman Chemical Co., Ann Arbor, Mich.) from differentiated THP-1 cells treated for 30 minutes with vehicle, LPS (100 ng/ml) or TNF-α (2 ng/ml) in the presence or absence of 10 μg/ml RTD-1 (SEQ ID NO. 1). NF-κB DNA binding was quantified by p65 ELISA (Cayman Chemical Co.). Non-specific DNA-binding, positive control extract and positive control extract treated with competitor DNA control included with the ELISA were used to confirm specificity of DNA-binding.

Isolation of Monocytes from Blood.

EDTA-anticoagulated blood was incubated with human monocyte enrichment cocktail (Stem Cell Technologies, Vancouver, BC, Canada) for 20 minutes and layered on Ficoll Paque™ in a Sepmate™ 50 tube (Stem Cell Tech). The cells were centrifuged at 1200×g for 10 minutes and the monocyte containing layer of cells was isolated, washed with PBS containing 2% HI FBS and 1 mM EDTA.

Immunoblot Analyses.

Differentiated THP-1 cells (˜8×10⁵ cells/2.5 ml medium) were starved in complete medium containing 1 FBS for 1 day as above, and fresh 2.5 ml RPMI 1640+1% FBS+P/S was added for 2 hours. For experiments with fresh monocytes, cells (>80% CD14+) were resuspended (˜10⁶ cells/nil) in monocyte attachment medium (Promocell, Heidelberg, Germany) in wells of a 12 well plate for 2 hours at 37° C. in 5% CO₂. The cells were then washed two times with RPMI medium containing 1% HI FBS and suspended overnight in RPMI medium containing 1% HI FBS. Cells were stimulated with agonists with or without RTD-1 (SEQ ID NO. 1) as described in the figure legends after which cells were washed with PBS and extracts were prepared in cell lysis buffer+1 mM PMSF. Protein content was determined using the BCA method (BioRad, San Diego, Calif.) and extracts resolved on SDS-tricine gels and transferred to nitrocellulose membrane. The membranes were probed with the indicated antibodies and developed using anti-mouse or anti-rabbit HRP conjugated secondary antibodies and detected by chemiluminescence. A phosphoprotein antibody array (Phospho-MAPK™; R&D systems Minneapolis, Minn.) was used to analyze activation of phosphoprotein kinases. Differentiated THP-1 cells (1.6×10⁶ cells) were treated with 100 ng/ml LPS, 10 μg/ml RTD-1 (SEQ ID NO. 1), 100 ng/ml LPS+10 μg/ml RTD-1, or vehicle for 30 minutes. Arrays were probed with 200 μg of cell extract protein and chemiluminescence of spots from arrays or bands from western blotting was quantified using NIH ImageJ software, background subtracted, normalized and plotted.

Statistical Analysis.

Standard deviation (SD) and standard error of mean (SEM) were calculated for experiments repeated 3 and 4 times respectively and Student's t-test was performed to evaluate the significance of peptide effects as indicated in the figure legends, difference was considered significant if P<0.05. Microsoft Excel was used for all statistical analyses.

RTD-1 (SEQ ID NO. 1) Suppression of Expression and Release of Pro-Inflammatory Cytokines.

Inventor(s) have found that RTD-1 (SEQ ID NO. 1) suppressed secretion of several inflammatory cytokines by peripheral blood leukocytes stimulated by agonists of TLRs 2, 4, 5, and 8 and the inhibition of TNF-α, IL-1β, and IL-8 release was most notable. RTD-1 (SEQ ID NO. 1) treatment markedly (>90%) suppressed secretion of TNF-α, IL-1β and IL-8 by LPS-stimulated primary human monocytes and THP-1 macrophages (see FIG. 1). RTD-1 (SEQ ID NO. 1) alone had no effect on THP-1 cytokine secretion (see FIG. 1). The cytokine inhibitory effects in THP-1 macrophages closely resemble the responses obtained with human blood buffy coat cells and human monocytes indicating that THP-1 cells provide an appropriate model for exploring RTD-1 (SEQ ID NO. 1)-mediated anti-inflammatory mechanisms.

In experiments similar to those described in FIG. 1, inventors analyzed the effect of RTD-1 (SEQ ID NO. 1) on cytokine mRNA expression by LPS-stimulated THP-1 cells, focusing on TNF-α, IL-1β, IL-8, CCL3, and CCL4. As shown in FIG. 2, RTD-1 (SEQ ID NO. 1) markedly inhibited LPS-induced mRNA expression of each of the 5 cytokines. The addition of RTD-1 (SEQ ID NO. 1) had no effect on the expression of GAPDH and the peptide alone had no effect on cytokine mRNA expression. These results (see FIGS. 1 and 2) demonstrate potent regulation of pro-inflammatory cytokine expression and release by RTD-1 (SEQ ID NO. 1) in LPS stimulated macrophages.

Regulation of NF-κB Pathway by RTD-1 (SEQ ID NO. 1).

Given the central role of NF-κB signaling in inflammation, inventors theorized that RTD-1 (SEQ ID NO. 1) modulates this pathway in THP-1 cells stimulated with different TLR agonists. LPS (TLR4 agonist) treatment of THP-1 cells induced a ˜7-fold activation of NF-κB (FIG. 3A). Simultaneous treatment with RTD-1 (SEQ ID NO. 1) and LPS suppressed NF-κB activation by ˜70%, whereas RTD-1 (SEQ ID NO. 1) alone had no effect (FIG. 3A). In addition, RTD-1 had no direct effect on the enzymatic activity of the SEAP reporter (LPS/RTD-1 (FIG. 3A); inhibition of NF-κB activation by RTD-1 (SEQ ID NO. 1) was dose dependent (FIG. 3B). Notably, no inhibition of NF-κB activity was observed when equimolar concentrations of human neutrophil α-defensin HNP-1 (FIG. 3B) or an acyclic form of RTD-1 (SEQ ID NO. 1) (S7; FIG. 3C) were used in place of RTD-1 (SEQ ID NO. 1).

To characterize the effects of RTD-1 on NF-κB activity induced by other stimuli, inventors analyzed responses of THP-1 cells stimulated with the TLR1/2 agonist Pam3CSK4 and HEK-Blue hTLR9 cells stimulated with TLR9 agonist ODN2006 with and without addition of RTD-1 (SEQ ID NO. 1). Both ligands stimulated NF-κB activation, 5- and 6-fold respectively. Addition of 10 μg/ml of RTD-1 inhibited Pam3CSK4-induced NF-κB activity by ˜70%, and the peptide suppressed ODN2006-mediated activation to baseline levels (FIGS. 3D and 3E, respectively).

Inventors further characterized the effect of RTD-1 (SEQ ID NO. 1) on LPS-stimulation of THP-1 cells by analyzing the effect of the peptide on DNA binding by NF-κB. LPS treatment induced a 4-fold increase in NF-κB DNA-binding (FIG. 4A). However, co-incubation of LPS and RTD-1 (SEQ ID NO. 1) reduced NF-κB DNA binding to baseline levels (FIG. 4A) which correlated with the levels of NF-κB p65 subunit levels in nuclear extracts from cells treated with LPS+/−RTD-1 (SEQ ID NO. 1) as detected by western blotting (FIG. 4A).

Inventors also tested the effect of RTD-1 (SEQ ID NO. 1) on TNF-α stimulation of NF-κB DNA binding. Compared with TNF-α alone, RTD-1 (SEQ ID NO. 1) suppressed NF-κB DNA binding by ˜35%, substantially less inhibition than observed in LPS-stimulated cells. The decrease in RTD-1 (SEQ ID NO. 1) inhibition of TNF-α-stimulated NF-κB DNA binding correlated with the levels of p65 in nuclear extracts of treated and untreated THP-1 cells (FIG. 4B).

The anti-inflammatory effects of RTD-1 (SEQ ID NO. 1) were compared to those of marimastat, a potent inhibitor of TNF-α converting enzyme (TACE/ADAM17), the sheddase that produces soluble TNF-α from the membrane bound precursor. Marimastat efficiently blocked release of TNF-α by LPS-stimulated THP-1 cells, but did not affect secretion of IL-1β (FIG. 5). This result is in contrast to the broader inhibitory activity of RTD-1 (SEQ ID NO. 1), which blocked both cytokines effectively (FIGS. 1 and 5A). Notably, marimastat treatment had no effect on LPS-stimulated NF-κB activation (FIG. 5B), in contrast to RTD-1 (SEQ ID NO. 1) which was a potent inhibitor of this activity in the presence or absence of marimastat (FIG. 5B).

Signaling Pathways Affected by RTD-1 (SEQ ID NO. 1).

LPS binding to TLR4 initiates a cascade of signaling events that leads to activation of a complex containing TGF-β-activated kinase 1 (TAK1) which phosphorylates IκB kinase (IKK). Activated IKK phosphorylates the NF-κB inhibitor IκBα triggering its degradation which activates NF-κB for nuclear translocation (FIG. 6). LPS-induced activation of TAK1 also stimulates numerous kinase pathways. RTD-1 (SEQ ID NO. 1) was effective in blocking the degradation of IκBα in LPS stimulated macrophages and this was accompanied by inhibition of p38 MAP kinase and JNK1/2 kinase phosphorylation. RTD-1 (SEQ ID NO. 1) effects when used alone were selective, showing no effect on IκBα levels or on phosphorylation of p38 MAP and JNK1/2 kinases (FIG. 6).

Inventors used phosphoprotein antibody arrays to identify other potential signaling targets of RTD-1 (SEQ ID NO. 1). Extracts of LPS-stimulated macrophages contained elevated levels of phosphorylated CREB, ERK2, H5P27, JNK2, p38α and p38γ compared to control cells (FIG. 7). RTD-1 (SEQ ID NO. 1) inhibited phosphorylation of each of these proteins to baseline levels (FIG. 7, lower panel) in such stimulated cells, however treatment with RTD-1 (SEQ ID NO. 1) alone did not suppress the phosphorylation of these proteins (FIG. 7).

In contrast, RTD-1 (SEQ ID NO. 1) alone or in combination with LPS selectively stimulated phosphorylation of Akt1 (FIG. 7). The kinetics of RTD-1 (SEQ ID NO. 1) effects on Akt expression and phosphorylation were determined by treating THP-1 cells with RTD-1 (SEQ ID NO. 1) alone or vehicle and analyzing Akt and P-Akt levels. RTD-1 (SEQ ID NO. 1) treatment induced a 6.5 fold increase in P-Akt within 10 minutes of peptide treatment and P-Akt levels remained elevated by ˜2.5 fold for at least 4 hours (FIG. 8A). Additional experiments were performed to identify the role of PI3K in the observed RTD-1-dependent stimulation of Akt phosphorylation. Addition of PI3K inhibitor Ly294002 markedly reduced RTD-1 (SEQ ID NO. 1) induced P-Akt levels (FIG. 8B) demonstrating that RTD-1 effect was upstream of Akt. Because the PI3K-Akt pathway negatively regulates activation of NF-κB and MAP kinase pathways, inventors characterized the effect of Ly294002 on the RTD-1 mediated inhibition of NF-κB activation in LPS stimulated THP-1 Dual macrophage cells. Ly294002 reversed the suppression of NF-κB activation by RTD-1 (SEQ ID NO. 1) in dose-dependent manner (FIG. 8C), evidence that stimulation of Akt phosphorylation contributes to the observed anti-inflammatory effects of RTD-1 (SEQ ID NO. 1).

It should be appreciated that the effects of θ-defensin and/or θ-defensin show a high degree of selectivity, exerting effects selectively to cells exposed to inflammatory stimuli and specifically impacting on certain processes and/or pathways in such cells. As such, treatment with θ-defensin and/or θ-defensin can provide selective therapy, particularly in immune-compromised or potentially immune-compromised individuals. Such therapy can effectively reduce expression of pro-inflammatory peptides in an individual while not inducing an immunocompromised state (i.e. not producing an immunocompromised state in a non-immunocompromised individual and/or not exacerbating the degree of immunocompromise in an immunocompromised individual).

Inventors also characterized the effects of RTD-1 (SEQ ID NO. 1) on signaling pathways in primary human monocytes. RTD-1 (SEQ ID NO. 1) alone did not induce degradation of IκBα but it reduced the degradation of IκBα in LPS stimulated monocytes (FIG. 9). RTD-1 (SEQ ID NO. 1) inhibited p38 MAP kinase phosphorylation in LPS stimulated monocytes, but the peptide alone had no effect on p38 MAP kinase phosphorylation (FIG. 9). However, as observed in experiments with THP-1 macrophages, increased phosphorylation of Akt was observed in monocytes treated with LPS, LPS and RTD-1 (SEQ ID NO. 1) or with RTD-1 (SEQ ID NO. 1) alone compared to control cells (FIG. 9). Thus, RTD-1 (SEQ ID NO. 1) regulates NF-κB, MAP kinase and PI3K-Akt signaling pathways across a range of cell types, including human blood monocytes and THP-1 cells.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

### REFERENCES

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.
1. Ericksen, B., Wu, Z., Lu, W., Lehrer, R. I. (2005) Antibacterial activity and specificity of the six human α-defensins. Antimicrob Agents Chemother 49, 269-275.
2. Mambula, S. S., Simons, E. R., Hastey, R., Selsted, M. E., Levitz, S. M. (2000) Human neutrophil-mediated nonoxidative antifungal activity against Cryptococcus neoformans. Infect Immun 68, 6257-6264.
3. Newman, S. L., Gootee, L., Gabay, J. E., Selsted, M. E. (2000) Identification of constituents of human neutrophil azurophil granules that mediate fungistasis against Histoplasma capsulatum. Infect Immun 68, 5668-5672.
4. Aley, S. B., Zimmerman, M., Hetsko, M., Selsted, M. E., Gillin, F. D. (1994) Killing of Giardia lamblia by cryptdins and cationic neutrophil peptides. Infect Immun 62, 5397-5403.
5. Daher, K. A., Selsted, M. E., Lehrer, R. I. (1986) Direct inactivation of viruses by human granulocyte defensins. J Virol 60, 1068-1074.
6. Bastian, A. and Schafer, H. (2001) Human α-defensin 1 (HNP-1) inhibits adenoviral infection in vitro. Regul Pept 101, 157-161.
7. Khine, A. A., Del Sorbo, L., Vaschetto, R., Voglis, S., Tullis, E., Slutsky, A. S., Downey, G. P., Zhang, H. (2006) Human neutrophil peptides induce interleukin-8 production through the P2Y6 signaling pathway. Blood 107, 2936-2942.
8. Boniotto, M., Jordan, W. J., Eskdale, J., Tossi, A., Antcheva, N., Crovella, S., Connell, N. D., Gallagher, G. (2006) Human β-defensin 2 induces a vigorous cytokine response in peripheral blood mononuclear cells. Antimicrob Agents Chemother 50, 1433-1441.
9. Ito, T., Tanabe, H., Ayabe, T., Ishikawa, C., Inaba, Y., Maemoto, A., Kono, T., Ashida, T., Fujiya, M., Kohgo, Y. (2012) Paneth cells regulate both chemotaxis of immature dendritic cells and cytokine production from epithelial cells. Tohoku J Exp Med 227, 39-48.
10. Yin, L., Chino, T., Horst, O. V., Hacker, B. M., Clark, E. A., Dale, B. A., Chung, W. O. (2010) Differential and coordinated expression of defensins and cytokines by gingival epithelial cells and dendritic cells in response to oral bacteria. BMC Immunol 11, 37.
11. Niyonsaba, F., Ushio, H., Nagaoka, I., Okumura, K., Ogawa, H. (2005) The human β-defensins (-1, -2, -3, -4) and cathelicidin LL-37 induce IL-18 secretion through p38 and ERK MAPK activation in primary human keratinocytes. J Immunol 175, 1776-1784.
12. Li, J., Zhu, H. Y., Beuerman, R. W. (2009) Stimulation of specific cytokines in human conjunctival epithelial cells by defensins HNP1, HBD2, and HBD3. Invest Ophthalmol Vis Sci 50, 644-653.
13. Syeda, F., Liu, H. Y., Tullis, E., Liu, M., Slutsky, A. S., Zhang, H. (2008) Differential signaling mechanisms of HNP-induced IL-8 production in human lung epithelial cells and monocytes. J Cell Physiol 214, 820-827.
14. Chertov, O., Michiel, D. F., Xu, L., Wang, J. M., Tani, K., Murphy, W. J., Longo, D. L., Taub, D. D., Oppenheim, J. J. (1996) Identification of defensin-1, defensin 2, and CAP37/azurocidin as T-cell chemoattractant proteins released from interleukin-8-stimulated neutrophils. J Biol Chem 271, 2935-2940.
15. Yang, D., Chertov, O., Bykovskaia, S. N., Chen, Q., Buffo, M. J., Shogan, J., Anderson, M., Schroder, J. M., Wang, J. M., Howard, O. M., Oppenheim, J. J. (1999) β-defensins: linking innate and adaptive immunity through dendritic and T cell CCR6. Science 286, 525-528.
16. Grigat, J., Soruri, A., Forssmann, U., Riggert, J., Zwirner, J. (2007) Chemoattraction of macrophages, T lymphocytes, and mast cells is evolutionarily conserved within the human α-defensin family. J Immunol 179, 3958-3965.
17. Soruri, A., Grigat, J., Forssmann, U., Riggert, J., Zwirner, J. (2007) β-Defensins chemoattract macrophages and mast cells but not lymphocytes and dendritic cells: CCR6 is not involved. Eur J Immunol 37, 2474-2486.
18. Selsted, M. E. and Ouellette, A. J. (2005) Mammalian defensins in the antimicrobial immune response. Nat Immunol 6, 551-557.
19. Lehrer, R. I., Cole, A. M., Selsted, M. E. (2012) theta-Defensins: cyclic peptides with endless potential. J Biol Chem 287, 27014-27019.
20. Tang, Y. Q., Yuan, J., Osapay, G., Osapay, K., Tran, D., Miller, C. J., Ouellette, A. J., Selsted, M. E. (1999) A cyclic antimicrobial peptide produced in primate leukocytes by the ligation of two truncated α-defensins. Science 286, 498-502.
21. Leonova, L., Kokryakov, V. N., Aleshina, G., Hong, T., Nguyen, T., Zhao, C., Waring, A. J., Lehrer, R. I. (2001) Circular minidefensins and posttranslational generation of molecular diversity. J Leukoc Biol 70, 461-464.
22. Garcia, A. E., Osapay, G., Tran, P. A., Yuan, J., Selsted, M. E. (2008) Isolation, synthesis, and antimicrobial activities of naturally occurring θ-defensin isoforms from baboon leukocytes. Infect Immun 76, 5883-5891.
23. Tongaonkar, P., Tran, P., Roberts, K., Schaal, J., Osapay, G., Tran, D., Ouellette, A. J., Selsted, M. E. (2011) Rhesus macaque θ-defensin isoforms: expression, antimicrobial activities, and demonstration of a prominent role in neutrophil granule microbicidal activities. J Leukoc Biol 89, 283-290.
24. Nguyen, T. X., Cole, A. M., Lehrer, R. I. (2003) Evolution of primate θ-defensins: a serpentine path to a sweet tooth. Peptides 24, 1647-1654.
25. Yang, D., Biragyn, A., Hoover, D. M., Lubkowski, J., Oppenheim, J. J. (2004) Multiple roles of antimicrobial defensins, cathelicidins, and eosinophil-derived neurotoxin in host defense. Annu Rev Immunol 22, 181-215.
26. Schaal, J. B., Tran, D., Tran, P., Osapay, G., Trinh, K., Roberts, K. D., Brasky, K. M., Tongaonkar, P., Ouellette, A. J., Selsted, M. E. (2012) Rhesus macaque theta defensins suppress inflammatory cytokines and enhance survival in mouse models of bacteremic sepsis. PLoS One 7, e51337.
27. Wohlford-Lenane, C. L., Meyerholz, D. K., Perlman, S., Zhou, H., Tran, D., Selsted, M. E., McCray, P. B., Jr. (2009) Rhesus theta-defensin prevents death in a mouse model of severe acute respiratory syndrome coronavirus pulmonary disease. J Virol 83, 11385-11390.
28. Ganz, T., Selsted, M. E., Szklarek, D., Harwig, S. S., Daher, K., Bainton, D. F., Lehrer, R. I. (1985) Defensins. Natural peptide antibiotics of human neutrophils. J Clin Invest 76, 1427-1435.
29. Tongaonkar, P., Golji, A. E., Tran, P., Ouellette, A. J., Selsted, M. E. (2012) High fidelity processing and activation of the human α-defensin HNP1 precursor by neutrophil elastase and proteinase 3. PLoS One 7, e32469.
30. Lee, J., Mira-Arbibe, L., Ulevitch, R. J. (2000) TAK1 regulates multiple protein kinase cascades activated by bacterial lipopolysaccharide. J Leukoc Biol 68, 909-915.
31. Guha, M. and Mackman, N. (2002) The phosphatidylinositol 3-kinase-Akt pathway limits lipopolysaccharide activation of signaling pathways and expression of inflammatory mediators in human monocytic cells. J Biol Chem 277, 32124-32132.
32. Yasin, B., Wang, W., Pang, M., Cheshenko, N., Hong, T., Waring, A. J., Herold, B. C., Wagar, E. A., Lehrer, R. I. (2004) Theta defensins protect cells from infection by herpes simplex virus by inhibiting viral adhesion and entry. J Virol 78, 5147-5156.
33. Cole, A. M., Hong, T., Boo, L. M., Nguyen, T., Zhao, C., Bristol, G., Zack, J. A., Waring, A. J., Yang, O. O., Lehrer, R. I. (2002) Retrocyclin: a primate peptide that protects cells from infection by T- and M-tropic strains of HIV-1. Proc Natl Acad Sci USA 99, 1813-1818.
34. Seidel, A., Ye, Y., de Armas, L. R., Soto, M., Yarosh, W., Marcsisin, R. A., Tran, D., Selsted, M. E., Camerini, D. (2010) Cyclic and acyclic defensins inhibit human immunodeficiency virus type-1 replication by different mechanisms. PLoS One 5, e9737.
35. Doss, M., Ruchala, P., Tecle, T., Gantz, D., Verma, A., Hartshorn, A., Crouch, E. C., Luong, H., Micewicz, E. D., Lehrer, R. I., Hartshorn, K. L. (2012) Hapivirins and diprovirins: novel θ-defensin analogs with potent activity against influenza A virus. J Immunol 188, 2759-2768.
36. Welkos, S., Cote, C. K., Hahn, U., Shastak, O., Jedermann, J., Bozue, J., Jung, G., Ruchala, P., Pratikhya, P., Tang, T., Lehrer, R. I., Beyer, W. (2011) Humanized θ-defensins (retrocyclins) enhance macrophage performance and protect mice from experimental anthrax infections. Antimicrob Agents Chemother 55, 4238-4250. 37. Hayden, M. S. and Ghosh, S. (2011) NF-kappaB in immunobiology. Cell Res 21, 223-244.
38. Newton, K. and Dixit, V. M. (2012) Signaling in innate immunity and inflammation. Cold Spring Harb Perspect Biol 4.
39. Mahtani, K. R., Brook, M., Dean, J. L., Sully, G., Saklatvala, J., Clark, A. R. (2001) Mitogen-activated protein kinase p38 controls the expression and posttranslational modification of tristetraprolin, a regulator of tumor necrosis factor alpha mRNA stability. Mol Cell Biol 21, 6461-6469.
40. Rutault, K., Hazzalin, C. A., Mahadevan, L. C. (2001) Combinations of ERK and p38 MAPK inhibitors ablate tumor necrosis factor-α (TNF-α) mRNA induction. Evidence for selective destabilization of TNF-α transcripts. J Biol Chem 276, 6666-6674.
41. Winzen, R., Kracht, M., Ritter, B., Wilhelm, A., Chen, C. Y., Shyu, A. B., Muller, M., Gaestel, M., Resch, K., Holtmann, H. (1999) The p38 MAP kinase pathway signals for cytokine-induced mRNA stabilization via MAP kinase-activated protein kinase 2 and an AU-rich region-targeted mechanism. Embo J 18, 4969-4980.
42. Wang, S. W., Pawlowski, J., Wathen, S. T., Kinney, S. D., Lichenstein, H. S., Manthey, C. L. (1999) Cytokine mRNA decay is accelerated by an inhibitor of p38-mitogen-activated protein kinase. Inflamm Res 48, 533-538.
43. Holtmann, H., Winzen, R., Holland, P., Eickemeier, S., Hoffmann, E., Wallach, D., Malinin, N. L., Cooper, J. A., Resch, K., Kracht, M. (1999) Induction of interleukin-8 synthesis integrates effects on transcription and mRNA degradation from at least three different cytokine- or stress-activated signal transduction pathways. Mol Cell Biol 19, 6742-6753.
44. Frevel, M. A., Bakheet, T., Silva, A. M., Hissong, J. G., Khabar, K. S., Williams, B. R. (2003) p38 Mitogen-activated protein kinase-dependent and independent signaling of mRNA stability of AU-rich element-containing transcripts. Mol Cell Biol 23, 425-436.
45. Dumitru, C. D., Ceci, J. D., Tsatsanis, C., Kontoyiannis, D., Stamatakis, K., Lin, J. H., Patriotis, C., Jenkins, N. A., Copeland, N. G., Kollias, G., Tsichlis, P. N. (2000) TNF-α induction by LPS is regulated posttranscriptionally via a Tpl2/ERK-dependent pathway. Cell 103, 1071-1083.
46. Karin, M. (2005) Inflammation-activated protein kinases as targets for drug development. Proc Am Thorac Soc 2, 386-390.
47. Park, Y. C., Lee, C. H., Kang, H. S., Chung, H. T., Kim, H. D. (1997) Wortmannin, a specific inhibitor of phosphatidylinositol-3-kinase, enhances LPS-induced NO production from murine peritoneal macrophages. Biochem Biophys Res Commun 240, 692-696.
48. Fukao, T. and Koyasu, S. (2003) PI3K and negative regulation of TLR signaling. Trends Immunol 24, 358-363.
49. Zong, Y., Sun, L., Liu, B., Deng, Y. S., Zhan, D., Chen, Y. L., He, Y., Liu, J., Zhang, Z. J., Sun, J., Lu, D. (2012) Resveratrol inhibits LPS-induced MAPKs activation via activation of the phosphatidylinositol 3-kinase pathway in murine RAW 264.7 macrophage cells. PLoS One 7, e44107.
50. Schabbauer, G., Tencati, M., Pedersen, B., Pawlinski, R., Mackman, N. (2004) PI3K-Akt pathway suppresses coagulation and inflammation in endotoxemic mice. Arterioscler Thromb Vasc Biol 24, 1963-1969.
51. Williams, D. L., Li, C., Ha, T., Ozment-Skelton, T., Kalbfleisch, J. H., Preiszner, J., Brooks, L., Breuel, K., Schweitzer, J. B. (2004) Modulation of the phosphoinositide 3-kinase pathway alters innate resistance to polymicrobial sepsis. J Immunol 172, 449-456.
52. Rohrl, J., Yang, D., Oppenheim, J. J., Hehlgans, T. (2010) Human beta-defensin 2 and 3 and their mouse orthologs induce chemotaxis through interaction with CCR2. J Immunol 184, 6688-6694.
53. Semple, F., MacPherson, H., Webb, S., Cox, S. L., Mallin, L. J., Tyrrell, C., Grimes, G. R., Semple, C. A., Nix, M. A., Millhauser, G. L., Dorin, J. R. (2011) Human β-defensin 3 affects the activity of pro-inflammatory pathways associated with MyD88 and TRIF. Eur J Immunol 41, 3291-3300.
54. Semple, F., Webb, S., Li, H. N., Patel, H. B., Perretti, M., Jackson, I. J., Gray, M., Davidson, D. J., Dorin, J. R. (2010) Human β-defensin 3 has immunosuppressive activity in vitro and in vivo. Eur J Immunol 40, 1073-1078.
55. Zheng, Y., Niyonsaba, F., Ushio, H., Nagaoka, I., Ikeda, S., Okumura, K., Ogawa, H. (2007) Cathelicidin LL-37 induces the generation of reactive oxygen species and release of human alpha-defensins from neutrophils. Br J Dermatol 157, 1124-1131.
56. Kahlenberg, J. M. and Kaplan, M. J. (2013) Little peptide, big effects: the role of LL-37 in inflammation and autoimmune disease. J Immunol 191, 4895-4901.
57. Mookherjee, N., Brown, K. L., Bowdish, D. M., Doria, S., Falsafi, R., Hokamp, K., Roche, F. M., Mu, R., Doho, G. H., Pistolic, J., Powers, J. P., Bryan, J., Brinkman, F. S., Hancock, R. E. (2006) Modulation of the TLR-mediated inflammatory response by the endogenous human host defense peptide LL-37. J Immunol 176, 2455-2464.

1. A method for modulation of multiple pro-inflammatory peptides in a human or nonhuman mammal, comprising administering a θ-defensin or θ-defensin analog to a subject in need thereof, in an amount effective to modulate a plurality of pro-inflammatory peptides by suppressing gene expression of the pro-inflammatory peptides.

2. The method of claim 1, wherein the θ-defensin is selected from the group consisting of RTD-1 (SEQ ID NO. 1), RTD-2 (SEQ ID NO. 5), RTD-3 (SEQ ID NO. 6), RTD-4 (SEQ ID NO. 7), RTD-5 (SEQ ID NO. 8), and RTD-6 (SEQ ID NO. 9).

3. The method of claim 1, wherein the θ-defensin analog is selected from the group consisting of RTD 1-27 (SEQ ID NO. 2), RTD 1-28 (SEQ ID NO. 3) and RTD 1-29 (SEQ ID NO. 4).

4. The method of claim 1 wherein the modulation of injurious cytokines or chemokines is mediated by suppression NF-κB signaling by the macrocyclic peptide.

5. The method of claim 1 wherein the modulation of the pro-inflammatory peptides is mediated by suppression of MAP kinase activation by the θ-defensin or θ-defensin analog.

6. The method of claim 1 wherein the modulation of the pro-inflammatory peptides is a result of activation of the PI3K/Akt pathway by the θ-defensin or θ-defensin analog.

7. The method of claim 1 wherein modulation of the pro-inflammatory peptides is a result of suppression of an immune response induced by interaction between a Toll-like receptor agonist and a receptor for the Toll-like receptor agonist.

8. The method of claim 1 wherein the θ-defensin or θ-defensin analog interrupts autocrine or paracrine proinflammatory signaling by a cytokines or a chemokine.

9. The method of claim 1 wherein the θ-defensin or θ-defensin analog suppresses a proinflammatory MAP kinase.

10. The method of claim 9, wherein the proinflammatory MAP kinase is selected from the group consisting of JNK, p38 MAP kinase, and ERK.

11. The method of claim 1 wherein the θ-defensin or θ-defensin analog suppresses proinflammatory effects of the NF-κB pathway by inducing phosphorylation of Akt.

12. A method for suppressing a plurality pro-inflammatory peptides without producing an immunocompromised state, comprising administering a θ-defensin or θ-defensin analog, to a human or nonhuman subject in need thereof, in an amount effective to suppress the plurality of injurious cytokines or chemokines and that is also ineffective in inducing the immunocompromised state.

13. A method for suppressing a pro-inflammatory peptide without producing an immunocompromised state in the setting of infection comprising administering a θ-defensin or θ-defensin analog, to an infected human or nonhuman subject in need thereof, in an amount effective to suppress the plurality of injurious cytokines or chemokines and that is also ineffective in inducing the immunocompromised state.

14. A method for suppressing a plurality of pro-inflammatory peptides without producing an immunocompromised state in a setting of immunosuppression, comprising administering a θ-defensin or θ-defensin analog, to an immunosuppressed human or nonhuman subject in need thereof, in an amount effective to suppress the plurality of injurious cytokines or chemokines and that is also ineffective in inducing the immunocompromised state.

15. A method for suppressing a plurality of pro-inflammatory peptides without producing an immunocompromised state in a setting of autoimmune disease, comprising administering a θ-defensin or θ-defensin analog, to a human or nonhuman subject in need thereof and having an autoimmune disease, in an amount effective to suppress the plurality of injurious cytokines or chemokines and that is also ineffective in inducing the immunocompromised state.

16. The methods of any one of claims 12 to 15, wherein at least one of the plurality of pro-inflammatory peptides is selected from the group consisting of tumor necrosis factor alpha (TNFα), IL-1β, IL-6, IL-8, CCL3, and CCL4.

17. A composition for modulation of multiple pro-inflammatory peptides in a human or nonhuman mammal, comprising a θ-defensin or θ-defensin analog to a subject in need thereof, in an amount effective to modulate a plurality of pro-inflammatory peptides by suppressing gene expression of the pro-inflammatory peptides.

18. The composition of claim 17, wherein the θ-defensin is selected from the group consisting of RTD-1 (SEQ ID NO. 1), RTD-2 (SEQ ID NO. 5), RTD-3 (SEQ ID NO. 6), RTD-4 (SEQ ID NO. 7), RTD-5 (SEQ ID NO. 8), and RTD-6 (SEQ ID NO. 9).

19. The composition of claim 17, wherein the θ-defensin analog is selected from the group consisting of RTD 1-27 (SEQ ID NO. 2), RTD 1-28 (SEQ ID NO. 3) and RTD 1-29 (SEQ ID NO. 4).

20. The composition of claim 17 wherein the modulation of injurious cytokines or chemokines is mediated by suppression NF-κB signaling by the macrocyclic peptide.

21. The composition of claim 17 wherein the modulation of the pro-inflammatory peptides is mediated by suppression of MAP kinase activation by the θ-defensin or θ-defensin analog.

22. The composition of claim 17 wherein the modulation of the pro-inflammatory peptides is a result of activation of the PI3K/Akt pathway by the θ-defensin or θ-defensin analog.

23. The composition of claim 17 wherein modulation of the pro-inflammatory peptides is a result of suppression of an immune response induced by interaction between a Toll-like receptor agonist and a receptor for the Toll-like receptor agonist.

24. The composition of claim 17 wherein the θ-defensin or θ-defensin analog interrupts autocrine or paracrine proinflammatory signaling by a cytokines or a chemokine.

25. The composition of claim 17 wherein the θ-defensin or θ-defensin analog suppresses a proinflammatory MAP kinase.

26. The composition of claim 25, wherein the proinflammatory MAP kinase is selected from the group consisting of JNK, p38 MAP kinase, and ERK.

27. The composition of claim 17 wherein the θ-defensin or θ-defensin analog suppresses proinflammatory effects of the NF-κB pathway by inducing phosphorylation of Akt.


SEQUENCE LISTING (9 sequences)

SEQ ID NO: 1 - protein, 18 aa, Macaca mulatta
  PEPTIDE (1)..(18): RTD-1, cyclic peptide
GFCRCLCRRGVCRCICTR

SEQ ID NO: 2 - protein, 14 aa, Artificial Sequence
  Synthettic cyclic tetradecapeptide RTD-1-27
GFCRCRRGVCRCTR

SEQ ID NO: 3 - protein, 14 aa, Artificial Sequence
  Synthetic cyclic tetradecapeptide RTD-1-28
GVCIVRRRFCLCRR

SEQ ID NO: 4 - protein, 14 aa, Artificial Sequence
  Synthetic cyclic tetradecapeptide RTD-1-29
GVCLCIRGRCRCRR

SEQ ID NO: 5 - protein, 18 aa, Macaca mulatta
  PEPTIDE (1)..(18): RTD-2, cyclic peptide
GVCRCLCRRGVCRCLCRR

SEQ ID NO: 6 - protein, 18 aa, Macaca mulatta
  PEPTIDE (1)..(18): RTD-3, cyclic peptide
GFCRCICTRGFCRCICTR

SEQ ID NO: 7 - protein, 18 aa, Macaca mulatta
  PEPTIDE (1)..(18): RTD-4, cyclic peptide
GICRCICTRGFCRCICVL

SEQ ID NO: 8 - protein, 18 aa, Macaca mulatta
  PEPTIDE (1)..(18): RTD-5, cyclic peptide
GICRCLCRRGVCRCICVL

SEQ ID NO: 9 - protein, 18 aa, Macaca mulatta
  PEPTIDE (1)..(18): RTD-6, cyclic peptide
GICRCICVLGICRCICVL